(19) United States
(12) Patent Application Publication
(10) Pub. No.: US 2006/0282051 A1
(43) Pub. Date: Dec. 14, 2006

(54) Surgical towel having radiopaque element and methods for making same

(76) Inventors: Susan Reichheld, Toronto (CA); Paul C. Reichheld, Ottawa (CA)

Correspondence Address: RATNERPRESTIA, P O BOX 980, VALLEY FORGE, PA, 19482-0980, US

(21) Appl. No.: 11/151,364

(22) Filed: Jun. 13, 2005

(51) Int. Cl. A61B 19/00

(52) U.S. Cl. 604/362; 604/357

(57) ABSTRACT

A surgical towel useful in surgical applications. The surgical towel has opposed surfaces and a plurality of edges. At least one pair of edges that are adjacent to one another are folded inwardly and secured to a surface to entirely enclose and retain a rigid radiopaque detection element at a corner of the towel. When so retained, a first portion of the detection element extends partially along a first edge of the pair of adjacent edges and a second portion of the detection element extends partially along a second edge of the pair of adjacent edges. The detection element is sized and shaped to inhibit potential injury to a patient being caused by the detection element if the surgical towel is temporarily left in a surgical cavity. Multiple detection elements may be so enclosed and retained.

### FIELD OF THE INVENTION

The present invention relates to substrates that are useful in surgical applications, and more particularly to surgical towels that are detectable in an X-ray image.

### BACKGROUND OF THE INVENTION

Surgical sponges are used in the course of surgery to absorb bodily fluids and also, after being soaked in saline, to temporarily pack and protect organs. Such sponges are not infrequently left behind in a patient's surgical cavity after an incision is closed.

Accordingly, procedures such as counting have been employed to prevent such occurrences. Counting, however, is an imperfect solution because it is susceptible to human error, a risk that is exacerbated when an unexpected emergency arises during the course of a surgical operation.

Therefore, counting procedures are supplemented by the use of surgical sponges having a radiopaque element associated therewith. After a surgical procedure has been completed, the patient is X-rayed, the theory being that, if a surgical sponge is inadvertently left within a patient's body cavity, the radiopaque element associated with that surgical sponge will be detectable in the X-ray image.

Numerous types of such surgical sponges are known. By way of example, U.S. Pat. No. 2,190,431 to Lewison discloses a surgical sponge in which a radiopaque glass thread is interwoven into the sponge's fibres. U.S. Pat. No. 2,698,270 to Mesek discloses a surgical sponge having a monofilament sealed onto a gauze pad in a wavy pattern, the monofilament comprised of an elastomer loaded with a radiopaque material. U.S. Pat. No. 3,133,538 to Pratt et al. discloses a surgical sponge having an associated radiopaque tracer thread wherein the thread is arranged in a series of loops. U.S. Pat. No. 3,508,551 to Walters et al. discloses a surgical dressing having a radiopaque filament locked in place by thermoplastic adhesion. Canadian Patent No. 1,056,100 to Rosenblatt discloses a medical sponge having an enclosed radiopaque substance distributed homogeneously throughout the material of the sponge. Canadian Patent No. 1,059,303 to Hardy et al. discloses a surgical sponge comprising an absorbent fabric and an X-ray detectable element integrally bonded to fibres in the fabric, wherein the X-ray detectable element has a configuration said to be recognizable in an X-ray image.

Canadian Patent No. 1,064,240 to McAvinn et al. discloses a surgical sponge comprising a sheet of absorbent material and an elongated visually detectable element at least partially located on an outer surface of the sheet, which element may also be radiopaque. Canadian Patent No. 1,229,962 to Rupinskas discloses a surgical sponge comprising a sheet of absorbent material made from entangled fibres, with the fibres being entangled about an elongated radiopaque element to retain the element in the sheet. Canadian Patent No. 1,249,122 to Jessup discloses a surgical sponge having an elongated visually detectable element comprising a pair of elongated twisted strands, one of which has a colour contrasting with the sponge and the other of which has a colour contrasting with blood, which strands may be radiopaque.

Alternatively, a separate radiopaque element may be associated with the sponge. Canadian Patent No. 1,293,601 to Dyer et al. discloses surgical swabs and sponges made from woven and nonwoven fabrics that are printed with a radiopaque polymeric composition that bonds to the surface of the fabric. U.S. Pat. No. 6,191,341 to Shippert discloses an absorbent pack for absorbing bodily fluids and which may be provided with a radiopaque marker.

Each of the types of sponge described above suffers from drawbacks. For example, a surgeon viewing an X-ray image can sometimes mistake the described radiopaque elements for tubes, ECG attachments or other external elements that might be present when the X-ray is taken. Moreover, if the surgical sponge becomes crumpled or bunched, the radiopaque elements may also become bunched and may be mistaken for ordinary tissue structures.

A surgical towel is to be distinguished from a surgical sponge. Surgical towels are generally used in surgical procedures to drape off an area of the patient's body adjacent to an incision, and to protect the tissue against instrumentation. As with surgical sponges, it is unfortunately not uncommon in the course of surgery for a surgical towel to enter a patient's open body cavity and then be left behind when the incision is closed, placing the patient's health at risk. Even where a surgical towel is large and is coloured to contrast with body tissues, the surgical towel can become saturated with blood or other fluids so that such contrast is reduced, and can become wadded up and be mistaken for a tissue structure upon visual inspection.

Attempts have been made to simply apply the methods used for surgical sponges to surgical towels. Canadian Patent Application Nos. 2,224,822 and 2,233,964 in the name of DeBusk disclose a fabric sheet having an elongated radiopaque element in the nature of a thread or strip of polyvinyl chloride containing barium sulphate. DeBusk discloses such a radiopaque element being captured within multiple folds along at least one edge of the fabric.

Unlike surgical sponges, which are used once and then discarded, surgical towels can be reused up to ten or more times if properly washed and sterilized. Such sterilization is typically performed using an autoclave, which applies blasts of steam to the surgical towel so that the heat destroys viral and bacterial contamination that may have been left behind after washing. The washing and autoclave process used to sterilize the surgical towel for reuse may cause damage to radiopaque elements conventionally used in surgical sponges, and such damage may increase each time the surgical towel is washed or sterilized. Therefore, a surgical towel that uses a radiopaque element similar to the type used in surgical sponges is not only subject to the disadvantage that the radiopaque element may be mistaken for something else, but may also be less likely to be visible in an X-ray image with each subsequent use.

### SUMMARY OF THE INVENTION

In one aspect, the present invention is directed to a surgical towel useful in surgical applications. The surgical towel has opposed surfaces and a plurality of edges. At least one pair of edges that are adjacent to one another are inwardly folded and secured to one surface to entirely enclose and retain a rigid radiopaque detection element at a corner of the towel. A first portion of the detection element extends partially along a first edge of the pair of adjacent edges and a second portion of the detection element extends partially along a second edge of the pair of adjacent edges. The detection element is sized and shaped to inhibit potential injury to a patient being caused by the detection element if the surgical towel is temporarily left in a surgical cavity.

In one embodiment, each detection element remains rigid and radiopaque after washing and exposure to blasts of steam, and is generally L-shaped, V-shaped or otherwise shaped to include two distal ends, such as two legs. The detection element may be made of stainless steel or other similar material with the requisite properties, and an end cap composed of material softer than the material from which the detection element is formed may be secured over each distal portion of the detection element. Each portion of each detection element may extend along its respective edge by a distance that is less than seven and one-half percent (7.5%) of the length of the respective edge and greater than five percent (5%) of the length of the respective edge.

In another embodiment, two pairs of adjacent edges are inwardly folded and secured to a surface to entirely enclose and retain two rigid radiopaque detection elements at two corners of the towel. A first portion of each detection element extends partially along a first edge of its respective pair of adjacent edges and a second portion of each detection element extends partially along a second edge of its respective pair of adjacent edges. Each detection element is sized and shaped to inhibit potential injury to a patient being caused by the detection element if the surgical towel is temporarily left in a surgical cavity. In one example of this embodiment, the two pairs of adjacent edges may share a common edge. In this particular embodiment, the detection elements remain rigid and radiopaque after washing and exposure to blasts of steam. The detection elements may generally be L-shaped, V-shaped or otherwise shaped to include distal ends such as legs. The detection elements may be made of stainless steel or other similar material with the requisite properties, and end caps composed of material softer than the material from which the detection elements are formed may be secured over each distal portion of each detection element. In this particular embodiment, each portion of each detection element may extend along its respective edge by a distance that is less than seven and one-half percent (7.5%) of the length of the respective edge and greater than five percent (5%) of the length of the respective edge.

In another aspect, a surgical towel has two opposed side edges, two opposed end edges and opposed surfaces. A first side edge and a first end edge are folded inwardly and secured to one surface to define a first side fold along the first side edge and a first side fold channel within the first side fold, and a first end fold along the first end edge and a first end fold channel within the first end fold. One of the first side fold or the first end fold partially overlaps and is secured to the other to form a first corner, and a rigid, radiopaque first detection element is retained at the first corner. The first detection element is entirely enclosed within the first side fold channel and the first end fold channel so that a first portion of the first detection element extends partially into the first side fold channel and a second portion of the first detection element extends partially into the first end fold channel. The first detection element is sized and shaped to inhibit potential injury to a patient being caused by the first detection element if the surgical towel is temporarily left in a surgical cavity.

In one embodiment, the first detection element remains rigid and radiopaque after being washed and exposed to blasts of steam, and may generally be L-shaped, V-shaped or otherwise shaped to include two distal ends such as legs. The detection element may be made of stainless steel or other similar material with the requisite properties, and an end cap composed of material softer than the material from which the detection element is formed may be secured over each distal portion of the detection element. In this embodiment, each portion of the first detection element may extend into its respective fold channel by a distance that may be less than seven and one-half percent (7.5%) of the length of the respective fold channel, and greater than five percent (5%) of the length of the respective fold channel.

In another embodiment, a second side edge is folded inwardly and secured to one surface to define a second side fold along the second side edge and a second side fold channel within the second side fold. One of the second side fold or the first end fold partially overlaps and is secured to the other to form a second corner. A rigid, radiopaque second detection element is retained at the second corner. The second detection element is entirely enclosed within the second side fold channel and the first end fold channel so that a first portion of the second detection element extends partially into the second side fold channel and a second portion of the second detection element extends partially into the first end fold channel. The second detection element is sized and shaped to inhibit potential injury to a patient being caused by the second detection element if the towel is temporarily left in a surgical cavity. In this embodiment, both detection elements remain rigid and radiopaque after being washed and exposed to blasts of steam, and may generally be L-shaped, V-shaped or otherwise shaped to include two distal ends, such as legs. The detection elements may be made of stainless steel or other similar material with the requisite properties, and end caps composed of material softer than the material from which the detection element is formed may be secured over each distal portion of each detection element. Also in this embodiment, each portion of each detection element may extend into its respective fold channel by a distance that may be less than seven and one-half percent (7.5%) of the length of the respective fold channel, and greater than five percent (5%) of the length of the respective fold channel.

In another embodiment, a second end edge is folded inwardly and secured to one surface to define a second end fold along the second end edge and a second end fold channel within the second end fold. One of the second end fold or the first side fold partially overlaps and is secured to the other to form a second corner. A rigid, radiopaque second detection element is retained at the second corner. The second detection element is entirely enclosed within the second end fold channel and the first side fold channel so that a first portion of the second detection element extends partially into the second end fold channel and a second portion of the second detection element extends partially into the first side fold channel. The second detection element is sized and shaped to inhibit potential injury to a patient being caused by the first detection element if the surgical towel is temporarily left in a surgical cavity. In this embodiment, both detection elements remain rigid and radiopaque after being washed and exposed to blasts of steam, and may generally be L-shaped, V-shaped or otherwise shaped to include two distal ends, such as legs. The detection elements may be made of stainless steel or other similar material with the requisite properties, and end caps composed of material softer than the material from which the detection elements are formed may be secured over each distal portion of each detection element. In this embodiment, each portion of each detection element may extend into its respective fold channel by a distance that may be less than seven and one-half percent (7.5%) of the length of the respective fold channel and greater than five percent (5%) of the length of the respective fold channel.

In another aspect, the present invention is directed to a method of making a surgical towel useful in surgical applications. According to the method, a sheet of fabric composed of material suitable for use in surgical applications and having opposed surfaces, a plurality of edges and a plurality of corners defined by pairs of adjacent edges, is provided. A first detection element is captured at a first corner of the sheet by folding first and second adjacent edges inwardly to define first and second adjacent folds, respectively, and securing the first and second adjacent folds to one surface of the sheet. This is done in such a way that a first portion of the first detection element extends partially along the first edge and within the first fold and a second portion of the first detection element extends partially along the second edge and within the second fold. As a result, the first detection element is retained by and entirely enclosed within the first and second folds. The first detection element is sized and shaped to inhibit potential injury to a patient being caused by the first detection element if the surgical towel is temporarily left in a surgical cavity.

In one embodiment of the above method, the second edge is folded so that the second fold partially overlaps the first fold and the second edge is also secured to the first fold.

In another embodiment, a second detection element is captured at a second corner of the sheet by folding inwardly a third edge adjacent to the first edge to define a third fold adjacent to the first fold and securing the third fold to the top surface of the sheet. This is done in such a way that a first portion of the second detection element extends partially along the first edge and within the first fold and a second portion of the second detection element extends partially along the third edge and within the third fold. As a result, the second detection element is retained by and entirely enclosed within the first and third folds. The second detection element is sized and shaped to inhibit potential injury to a patient being caused by the second detection element if the surgical towel is temporarily left in a surgical cavity. In this particular embodiment, the second and third edges are folded so that the second and third folds each partially overlap the first fold, and the second edge and the third edge are also secured to the first fold.

### BRIEF DESCRIPTION OF THE DRAWINGS

These and other features of the preferred embodiments of the invention will become more apparent in the following detailed description in which reference is made to the appended drawings wherein:

FIG. 1 is a perspective view of a surgical towel;

FIG. 2 is a cross-sectional view of the surgical towel of FIG. 1, taken along the line 1-1;

FIG. 3 is a perspective view of a detection element;

FIG. 4 is a front view of a sheet of fabric;

FIG. 5 is a front view of the sheet of fabric of FIG. 4, with two opposed edges thereof folded inwardly;

FIG. 6 is a front view of the sheet of fabric of FIG. 4, with two opposed edges thereof folded inwardly and two detection elements placed in the folds;

FIG. 7 is a front view of a surgical towel;

FIG. 8 is a front view of a sheet of fabric;

FIG. 9 is a front view of the sheet of fabric of FIG. 8, with a first edge thereof folded inwardly;

FIG. 10 is a front view of the sheet of fabric of FIG. 8, with two detection elements placed in the first fold and a further two opposed edges folded inwardly; and

FIG. 11 is a front view of a surgical towel.

### DESCRIPTION OF THE PREFERRED EMBODIMENTS

An embodiment of a substrate for use in surgical applications, namely a surgical towel according to one aspect of the present invention, is shown generally at 10. The surgical towel is useful in surgical applications, and is made from a suitable absorbent fabric, such as cotton.

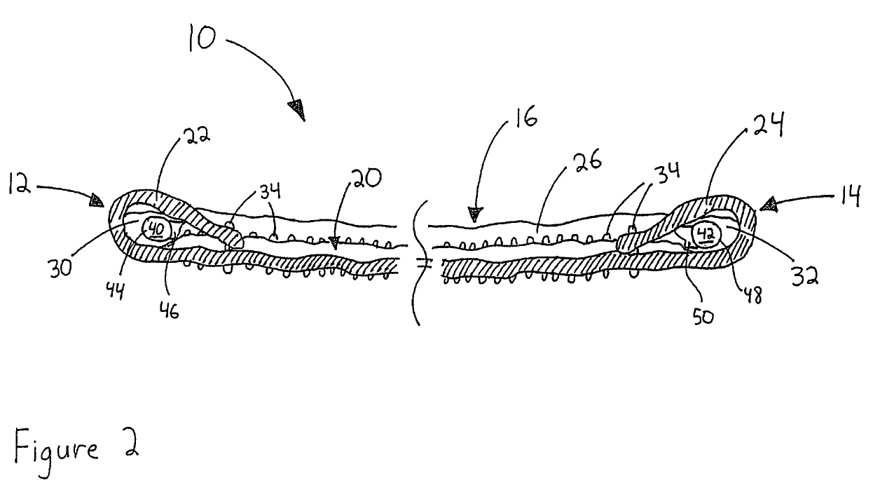

The surgical towel shown in the Figures has first and second side edges 12, 14, first and second end edges 16, 18, and atop surface 20. The side edges 12, 14 are folded inwardly and secured to the top surface 20 to form first and second side folds 22, 24 along the side edges, respectively. Similarly, the end edges 16, 18 are folded inwardly and secured to the top surface 20 to form respective first and second end folds 26, 28 along the end edges 16, 18. Each of the folds 22, 24, 26, 28 defines a fold channel therewithin. In FIG. 2, the side fold channels 30, 32 defined by the first and second side folds 22, 24, respectively, are shown. The edges 12, 14, 16, 18 are secured to the top surface 20 by stitching 34.

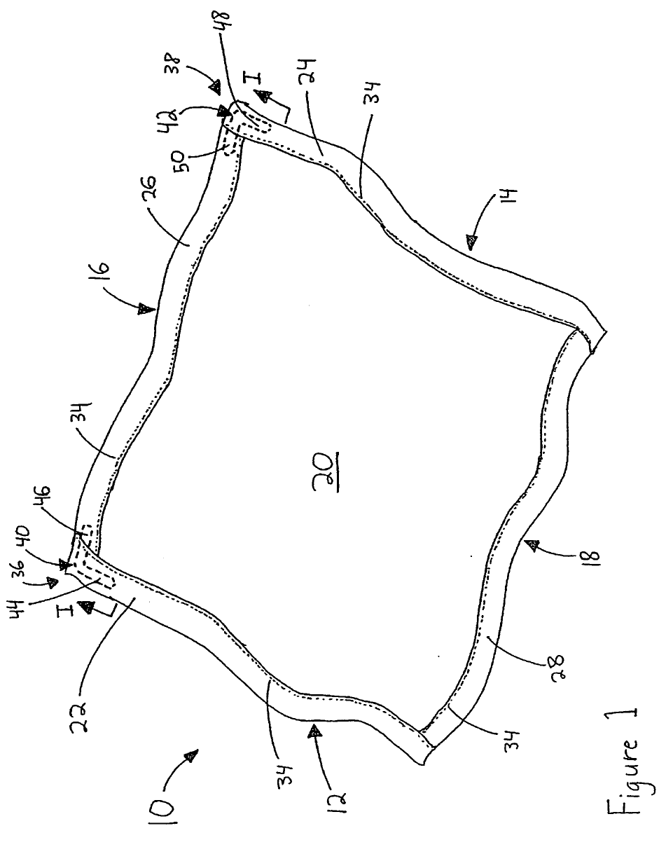

As may be seen in FIG. 1, the side folds 22, 24 partially overlap the end fold 26 to form corners 36, 38, respectively. A first rigid, radiopaque detection element 40 is retained at the corner 36, and a second rigid, radiopaque detection element 42 is retained at the corner 38. Each detection element 40, 42 respectively has a first portion 44, 48 and a second portion 46, 50. The first detection element 40 is entirely enclosed within the fold channel 30 formed by the first side fold 22 and the fold channel (not shown) formed by the first end fold 26. The first portion 44 of the first detection element 40 extends partially into the fold channel 30 formed by the first side fold 22 and the second portion 46 of the first detection element 40 extends partially into the fold channel (not shown) formed by the first end fold 26. The second detection element 42 is entirely enclosed within the fold channel 32 formed by the second side fold 24 and the fold channel (not shown) formed by the first end fold 26. The first portion 48 of the second detection element 42 extends partially into the fold channel 32 formed by the second side fold 24 and the second portion 50 also extends partially into the fold channel (not shown) formed by the first end fold 26.

As may be seen in FIG. 1, the first portion 44, 48 of each detection element 40, 42 extends partially along the first edge 12, 14 of a pair of adjacent edges and the second portion 46, 50 of each detection element 40, 42 extends partially along the second edge 16 of the pair of adjacent edges. Situating the detection element 40, 42 at the corners 36, 38 allows the surgical towel 10 to retain the flexibility necessary for it to be useful in surgical procedures. Each portion of the detection element 40, 42 extends far enough into its respective fold channel to facilitate retention of the detection element 40, 42 at its respective corner 36, 38, but not so far that the detection element 40, 42 substantially interferes with the flexibility of the side edges 12, 14 or the end edge 16. The first portions 44, 48 of each detection element 40, 42 extend into their respective fold channels 30, 32 by a distance equal to approximately seven and one-half percent (7.5%) of the length of the respective fold channel 30, 32. The second portions 46, 50 of each detection element 40, 42 extend into the fold channel (not shown) formed by the end fold 26 by a distance equal to approximately five percent (5%) of the length of the fold channel. Optionally, cross-stitching may be applied to the folds 22, 24, 26 adjacent the ends of the detection elements 40, 42 to close the fold channels 30, 32 and the fold channel (not shown) defined by the end fold 26. This will assist in retaining the detection elements 40, 42 at their respective corners 36, 38.

The surgical towel should be constructed in such a way as to avoid loose threads that can become separated from the surgical towel and enter the open surgical incision, where they can cause adverse effects. The possibility of loose threads at the edges of the fabric sheet used to construct the surgical towel can be obviated, for example, by the use of selvedge edges, namely where the edge of the fabric sheet is woven so that it will not fray or ravel. Alternatively, the folds 22, 24, 26, 28 may be double folded before stitching so that any loose threads are contained within the fold.

While the surgical towel has been shown and described in rectangular shape with two detection elements 40, 42, a surgical towel according to an aspect of the present invention may be of any suitable shape and may have only one detection element, or may have three or more detection elements. Moreover, where two detection elements are used, the detection elements may be placed at diagonally opposite corners, if desired. Also, while the surgical towel has been described as being composed of cotton, any flexible, absorbent fabric material that is suitable for contact with an open surgical incision of a patient may be used. Where it is intended to reuse the surgical towel, the surgical towel must be composed of a fabric material that can be washed and sterilized.

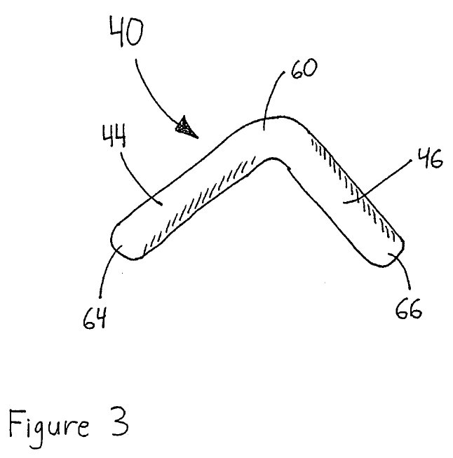

Referring specifically to FIG. 3, the detection element 40 has a generally circular cross-section and has a curved, generally L-shaped configuration. The detection element 40 comprises first portion 44 and second portion 46 and a generally curved elbow 60 disposed therebetween and which orients the portions 44, 46 so that they are substantially perpendicular to one another. The respective ends 64, 66 of the portions 44, 46 are rounded, as is the elbow 60, so that sharp or abrupt edges, which could wear down the material of the surgical towel or cause injury to a patient, are avoided.

The detection element 40 may be composed of stainless steel or any other similar material with the requisite properties. The element is radiopaque and will remain so even after being subjected to repeated washing and blasts of steam. Moreover, the detection element 40 is substantially rigid, so that it will not fold or bunch, and is shaped so as to be recognizable in an X-ray image.

If desired, end caps (not shown) formed of material softer than the material from which the detection element is formed may be placed over the ends 64, 66 of the detection element. This may reduce wear caused by rubbing of the ends 64, 66 against the material of the folds within which they are enclosed and thereby extend the useful life of the surgical towel.

The detection elements 40, 42 have been described as having portions that extend by a specified distance into the fold channels of the surgical towel. The portions of the detection elements may be of various lengths, provided that they extend far enough into their respective fold channels that the detection element is retained at its respective corner and not so far that the detection element substantially interferes with the flexibility of the surgical towel. Thus, where cross-stitching is used to close the fold channels of the surgical towel, the length of the portions of the detection elements may be shorter than when such cross-stitching is not used. The detection elements must also be sufficiently small that they will not cause injury to a patient if a surgical towel is inadvertently left in a patient's surgical cavity for a short period before being detected and removed. As noted above, the surfaces of the detection element 40 are rounded to reduce injury risk associated therewith. Thus, the detection element 40 is sized and shaped to inhibit potential injury to a patient being caused by the detection element 40 if the surgical towel 10 is temporarily left in a surgical cavity.

Substantially rigid detection elements for use in surgical towels according to an aspect of the present invention may be composed of radiopaque materials other than stainless steel. Such materials should be biologically non-adverse in the sense that the material will not harm a patient if a surgical towel having a detection element composed of such material is inadvertently left in a patient's surgical cavity for a short time before being detected and removed. If the surgical towel within which a detection element is enclosed is intended for a single use only, it is not necessary to construct the detection element from material that remains radiopaque after being subjected to washing and steam treatment.

The shape of the detection element 40 shown and described above is exemplary only. Detection elements for use in surgical towels according to an aspect of the present invention may have shapes other than that described above so long as they comprise shapes for extending into adjacent fold channels and are sized and shaped to inhibit potential injury to a patient being caused by the detection element 40 if the surgical towel 10 is temporarily left in a surgical cavity. Thus, any suitably shaped detection element having two distal portions, such as two legs, may be used.

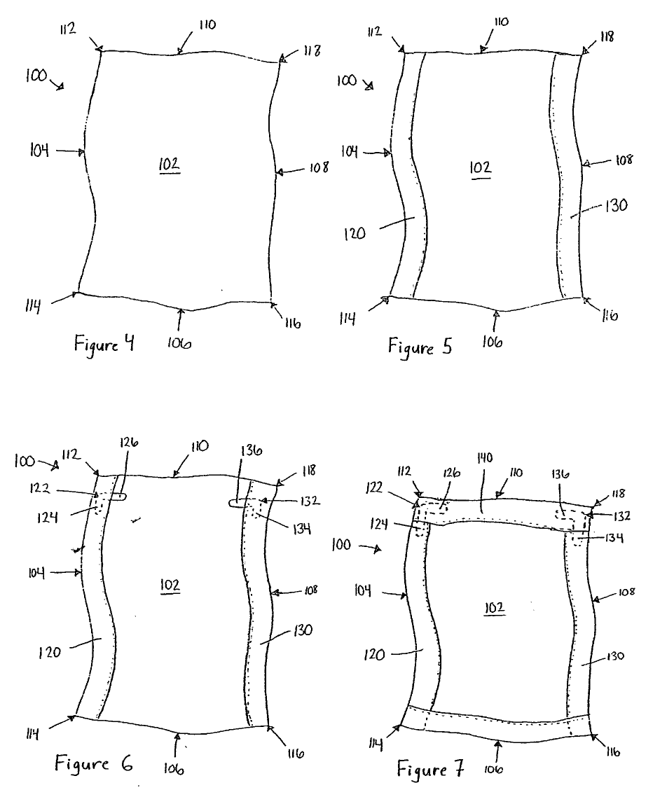

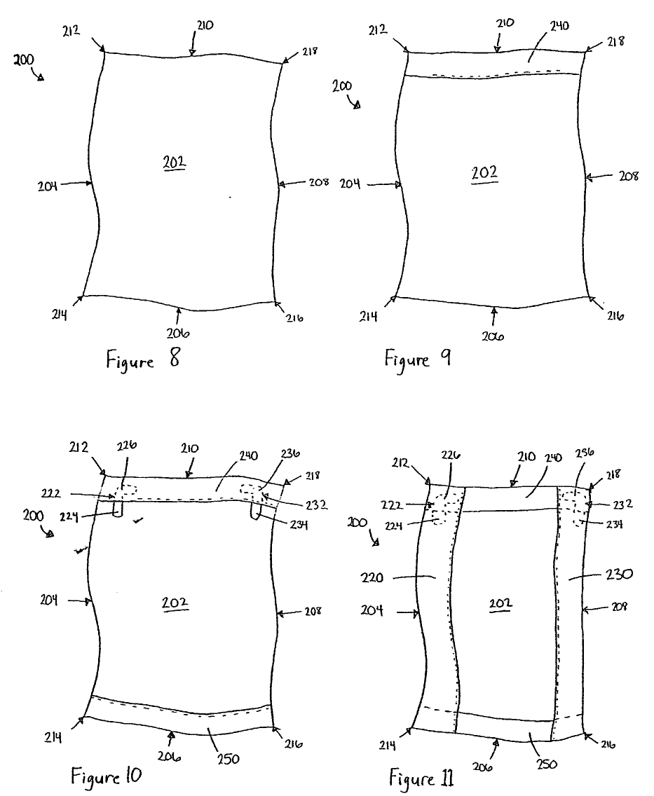

A surgical substrate of the present invention, such as the surgical towel described above, may be manufactured in various ways. One method is described with reference to FIGS. 4 to 7. A sheet of fabric 100 having one surface, in the Figures the top surface 102, a plurality of edges 104, 106, 108, 110 and a plurality of corners 112, 114, 116, 118 defined by pairs of adjacent edges and composed of material suitable for use in surgical applications is provided. A first edge 104 is folded inwardly to define a first fold 120, and a first detection element 122 is placed at a corner 112 of the sheet 100 so that a first portion 124 of the detection element 122 extends partially along the first edge 104 and within the first fold 120. A second edge 108 is folded inwardly to define a second fold 130, and a second detection element 132 is placed at a corner 118 of the sheet 100 so that a first portion 134 of the second detection element 132 extends partially along the second edge 108 and within the second fold 130. The first and second edges 104, 108 are then secured to the top surface 102 by stitching, leaving the portion of the respective edges 104, 108 extending from the respective second portions 126, 136 of the detection elements 120, 130 to the adjacent edge 110 unstitched.

The adjacent edge 110 is then folded inwardly to define a third fold 140 that overlaps the first and second folds 120, 130 so that the respective second portions 126, 136 of the detection elements 122, 132 extend partially along the adjacent edge 110 and within the third fold 140. The third fold 140 is then secured to the top surface 102 and to the first and second folds 120, 130 by stitching. As a result, the first detection element 122 is retained by and entirely enclosed within the first fold 120 and the third fold 140, and the second detection element 132 is retained by and entirely enclosed within the second fold 130 and the third fold 140. The fourth edge 106 is then folded inwardly so that it overlaps the first and second folds 120, 130 and secured to the top surface 102 by stitching to create a symmetric appearance.

Alternatively, a surgical towel may be manufactured in other ways. A second method is described with reference to FIGS. 8 to 11. A sheet of fabric 200 having opposed surfaces, including a top surface 202, a plurality of edges 204, 206, 208, 210 and a plurality of corners 212, 214, 216, 218 defined by pairs of adjacent edges and composed of material suitable for use in surgical applications is provided. A first edge 210 is folded inwardly to define a first fold 240, and a first detection element 222 is placed at a corner 212 of the sheet 200 so that a first portion 226 of the detection element 222 extends partially along the first edge 210 and within the first fold 240. A second detection element 232 is placed at a corner 218 of the sheet 200 so that a first portion 236 of the detection element 232 extends partially along the first edge 210 and within the first fold 240. The first edge 210 is then secured to the top surface 202 by stitching, leaving the ends extending from the respective second portions 224, 234 of the detection elements 222, 232 to the adjacent edges 204, 208 unstitched. The fourth edge 206 is then folded inwardly to define a second fold 250 and secured to the top surface 202 by stitching.

The adjacent edges 204 and 208 are then folded inwardly to define respective third and fourth folds 220, 230 that overlap the first fold 240 and the second fold 250. The second portion 224 of the detection element 222 extends partially along the edge 204 and within the third fold 220, and the second portion 234 of the detection element 232 extends partially along the edge 208 and within the fourth fold 230. The second and third folds 220, 230 are then secured to the top surface 202 and to the first fold 240 and the fourth fold 250 by stitching. As a result, the first detection element 222 is retained by and entirely enclosed within the first fold 240 and the third fold 220, and the second detection element 232 is retained by and entirely enclosed within the first fold 240 and the fourth fold 230.

The methods of constructing surgical towels described above refer to the exemplary case where two detection elements are included in each surgical towel. The methods may be suitably adapted to be used for the construction of surgical towels having only one detection element, or having three or more detection elements. In addition, while the step of stitching has been described above as occurring generally after each fold, it is envisioned that each stitching step may be done at a different stage than that described. For example, the methods described may be modified so that all folds are made and then all stitching is carried out in a single step. Moreover, depending on the material used, methods other than stitching may be used to secure the folds to the top surface. For example, where appropriate synthetic materials are used, the folds may be secured by thermal adhesion.

Although the invention has been described with reference to certain specific embodiments, various modifications thereof will be apparent to those skilled in the art without departing from the spirit and scope of the invention as outlined in the claims appended hereto.

The embodiments of the invention in which an exclusive property or privilege is claimed are defined as follows:

1. A surgical towel useful in surgical applications, the surgical towel comprising two surfaces, a plurality of edges, at least one pair of edges that are adjacent to one another being inwardly folded and secured to a first surface to entirely enclose and retain a rigid radiopaque detection element at a corner of the surgical towel so that a first portion of the detection element extends partially along a first edge of the pair of adjacent edges and a second portion of the detection element extends partially along a second edge of the pair of adjacent edges, wherein the detection element is sized and shaped to inhibit potential injury to a patient being caused by the detection element if the surgical towel is temporarily left in a surgical cavity.

2. The surgical towel of claim 1, wherein each detection element remains rigid and radiopaque after washing and exposure to blasts of steam.

3. The surgical towel of claim 1, wherein each detection element is generally L-shaped.

4. The surgical towel of claim 1, wherein each detection element is generally V-shaped.

5. The surgical towel of claim 1, wherein each detection element is shaped to include two distal portions.

6. The surgical towel of claim 2, wherein each detection element is composed of stainless steel.

7. The surgical towel of claim 5 wherein an end cap composed of material softer than the material from which the detection element is formed is secured over each distal portion of the detection element.

8. The surgical towel of claim 1, wherein each portion of each detection element extends along its respective edge by a distance that is less than seven and one-half percent (7.5%) of the length of the respective edge.

9. The surgical towel of claim 8, wherein each portion of each detection element extends along its respective edge by a distance that is greater than five percent (5%) of the length of the respective edge.

10. The surgical towel of claim 1, wherein a second pair of adjacent edges are folded inwardly and secured to the first surface to entirely enclose and retain a second rigid, radiopaque detection element at a second corner of the surgical towel so that a first portion of the second detection element extends partially along a first edge of the second pair of adjacent edges and a second portion of the second detection element extends partially along a second edge of the second pair of adjacent edges and wherein the second detection element is sized and shaped to inhibit potential injury to a patient being caused by the second detection element if the surgical towel is temporarily left in a surgical cavity.

11. The surgical towel of claim 10, wherein the two pairs of adjacent edges share a common edge.

12. The surgical towel of claim 10, wherein the detection elements remain rigid and radiopaque after washing and exposure to blasts of steam.

13. The surgical towel of claim 10, wherein the detection elements are generally L-shaped.

14. The surgical towel of claim 10, wherein the detection elements are generally V-shaped.

15. The surgical towel of claim 10, wherein the detection elements are shaped to include two distal portions.

16. The surgical towel of claim 12, wherein the detection elements are composed of stainless steel.

17. The surgical towel of claim 15 wherein end caps composed of material softer than the material from which the detection elements are formed are secured over each distal portion of each detection element.

18. The surgical towel of claim 10, wherein each portion of each detection element extends along its respective edge by a distance that is less than seven and one-half percent (7.5%) of the length of the respective edge.

19. The surgical towel of claim 18 wherein each portion of each detection element extends along its respective edge by a distance that is greater than five percent (5%) of the length of the respective edge.

20. A surgical towel useful in surgical applications, the surgical towel having two opposed side edges, two opposed end edges, and two surfaces, wherein a first side edge and a first end edge are folded inwardly and secured to a first surface to define a first side fold along the first side edge and a first side fold channel within the first side fold, and a first end fold along the first end edge and a first end fold channel within the first end fold, one of the first side fold or the first end fold partially overlapping and being secured to the other to form a first corner, wherein a rigid radiopaque first detection element is retained at the first corner, the first detection element being entirely enclosed within the first side fold channel and the first end fold channel so that a first portion of the first detection element extends partially into the first side fold channel and a second portion of the first detection element extends partially into the first end fold channel and wherein the first detection element is sized and shaped to inhibit potential injury to a patient being caused by the first detection element if the surgical towel is temporarily left in a surgical cavity.

21. The surgical towel of claim 20, wherein the first detection element remains rigid and radiopaque after being washed and exposed to blasts of steam

22. The surgical towel of claim 20, wherein the first detection element is generally L-shaped.

23. The surgical towel of claim 20, wherein the first detection element is generally V-shaped.

24. The surgical towel of claim 20, wherein the first detection element is shaped to include two distal portions.

25. The surgical towel of claim 20, wherein the first detection element is composed of stainless steel.

26. The surgical towel of claim 24 wherein an end cap composed of material softer than the material from which the first detection element is formed is secured over each distal portion of the first detection element.

27. The surgical towel of claim 20, wherein each portion of the first detection element extends into its respective fold channel by a distance that is less than seven and one-half percent (7.5%) of the length of the respective fold channel.

28. The surgical towel of claim 27, wherein each portion of the first detection element extends into its respective fold channel by a distance that is greater than five percent (5%) of the length of the respective fold channel.

29. The surgical towel of claim 20, wherein a second side edge is folded inwardly and secured to the first surface to define a second side fold along the second side edge and a second side fold channel within the second side fold, one of the second side fold or the first end fold partially overlapping and being secured to the other to form a second corner, wherein a rigid radiopaque second detection element is retained at the second corner, the second detection element being entirely enclosed within the second side fold channel and the first end fold channel so that a first portion of the second detection element extends partially into the second side fold channel and a second portion of the second detection element extends partially into the first end fold channel and wherein the second detection element is sized and shaped to inhibit potential injury to a patient being caused by the second detection element if the surgical towel is temporarily left in a surgical cavity.

30. The surgical towel of claim 29, wherein the detection elements remain rigid and radiopaque after being washed and exposed to blasts of steam.

31. The surgical towel of claim 29, wherein the detection elements are generally L-shaped.

32. The surgical towel of claim 29, wherein the detection elements are generally V-shaped.

33. The surgical towel of claim 29, wherein the detection elements are shaped to include two distal portions.

34. The surgical towel of claim 30, wherein the detection elements are composed of stainless steel.

35. The surgical towel of claim 33 wherein end caps composed of material softer than the material from which the detection elements are formed are secured over each distal portion of each detection element.

36. The surgical towel of claim 29, wherein each portion of each detection element extends into its respective fold channel by a distance that is less than seven and one-half percent (7.5%) of the length of the respective fold channel.

37. The surgical towel of claim 36, wherein each portion of each detection element extends into its respective fold channel by a distance that is greater than five percent (5%) of the length of the respective fold channel.

38. The surgical towel of claim 20, wherein a second end edge is folded inwardly and secured to the first surface to define a second end fold along the second end edge and a second end fold channel within the second end fold, one of the second end fold or the first side fold partially overlapping and being secured to the other to form a second corner, wherein a rigid radiopaque second detection element is retained at the second corner, the second detection element being entirely enclosed within the second end fold channel and the first side fold channel so that a first portion of the second detection element extends partially into the second end fold channel and a second portion of the second detection element extends partially into the first side fold channel and wherein the second detection element is sized and shaped to inhibit potential injury to a patient being caused by the second detection element if the surgical towel is temporarily left in a surgical cavity.

39. The surgical towel of claim 38, wherein the detection elements remain rigid and radiopaque after being washed and exposed to blasts of steam.

40. The surgical towel of claim 38, wherein the detection elements are generally L-shaped.

41. The surgical towel of claim 38, wherein the detection elements are generally V-shaped.

42. The surgical towel of claim 38, wherein the detection elements are shaped to include two distal portions.

43. The surgical towel of claim 39, wherein the detection elements are composed of stainless steel.

44. The surgical towel of claim 42 wherein end caps composed of material softer than the material from which the detection elements are formed are secured over each distal portion of each detection element.

45. The surgical towel of claim 38, wherein each portion of each detection element extends into its respective fold channel by a distance that is less than seven and one-half percent (7.5%) of the length of the respective fold channel.

46. The surgical towel of claim 45, wherein each portion of each detection element extends into its respective fold channel by a distance that is greater than five percent (5%) of the length of the respective fold channel.

47. A method of making a surgical towel useful in surgical applications, the method comprising the steps of:
(a) providing a sheet of fabric composed of material suitable for use in surgical applications, the sheet having two surfaces, a plurality of edges and a plurality of corners defined by pairs of adjacent edges; and
(b) capturing a first detection element at a first corner of the sheet by folding first and second adjacent edges inwardly to define first and second adjacent folds, respectively, and securing the first and second adjacent folds to a first surface of the sheet so that a first portion of the first detection element extends partially along the first edge and within the first fold and a second portion of the first detection element extends partially along the second edge and within the second fold and the first detection element is retained by and entirely enclosed within the first and second folds, wherein the first detection element is sized and shaped to inhibit potential injury to a patient being caused by the first detection element if the surgical towel is temporarily left in a surgical cavity.

48. The method of claim 47, wherein the second edge is folded so that the second fold partially overlaps the first fold.

49. The method of claim 48, wherein the second edge is also secured to the first fold.

50. The method of claim 47, further comprising the step of:
(c) capturing a second detection element at a second corner of the sheet by folding inwardly a third edge adjacent to the first edge to define a third fold adjacent to the first fold and securing the third fold to the first surface of the sheet so that a first portion of the second detection element extends partially along the first edge and within the first fold and a second portion of the second detection element extends partially along the third edge and within the third fold and the second detection element is retained by and entirely enclosed within the first and third folds, wherein the second detection element is sized and shaped to inhibit potential injury to a patient being caused by the second detection element if the surgical towel is temporarily left in a surgical cavity.

51. The method of claim 50, wherein the second and third edges are folded so that the second and third folds each partially overlap the first fold.

52. The method of claim 51, wherein the second edge and the third edge are also secured to the first fold.
